(19) United States
(12) Patent Application Publication
(10) Pub. No.: US 2016/0317573 A1
(43) Pub. Date: Nov. 3, 2016

(54) PROPHYLAXIS AND THERAPY FOR AGE RELATED ILLNESSES

(71) Applicant: Kumarpal A. Shah, Searingtown, NY (US)

(72) Inventor: Kumarpal A. Shah, Searingtown, NY (US)

(21) Appl. No.: 14/699,479

(22) Filed: Apr. 29, 2015

(51) Int. Cl. A61K 31/795

(52) U.S. Cl. CPC A61K 31/795

(57) ABSTRACT

A method for treating a subject suffering from age related illnesses such as Type 2 diabetes by administering to the subject a compound to down regulate Factor D or Factor H thereby providing prophylaxis and therapy to inhibit age related illnesses. The compound is a functionalized Nano Polymer of less than 100 nm, which primarily inhibits Factor D, Factor H, or both.

### BACKGROUND OF THE INVENTION

1. Field of the Invention

The prophylaxis and therapy of Type 2 Diabetes and its complications are detailed with Nano formulated sulfonic polymers and its formulation variations. This is a model example of immune pathogenesis of age related illnesses due to genetic and environmental interactions with Factor H.

2. The Prior Art

Rapid advances in aging research make it feasible to extend human life with a pill. In keeping with this enthusiasm, the recent issue of “TIME” published on Feb. 23/Mar. 2, 2015, explored the frontiers of Longevity and the prospect of human life extension up to 142 years (1).

Sharp Z. D. et al in U.S. Patent Application Publication U.S. 2012/0064143 A1 dated Mar. 15, 2012, details “Inhibition of Mammalian Target of Rapamycin”. The publication discloses a method for treating and preventing an age related disease, condition or disorder by microcapsule of rapamycin that is an inhibitor of the mammalian target of rapamycin (mTOR) (2).

Rapamycin by inhibiting mTOR pathway can extend lifespan and improve age-related functional decline in mice, thereby providing the first proof of principal that a pharmaceutical agent can slow the aging process in mammals. These outcomes have proven robust in repeated studies. Translational studies that assess rapamycin's effects on human aging and age-related disease are therefore thought to be within reach and have actually been initiated at some sites (ClinicalTrials.gov:NCT01649960).

Rapamycin is an immune modulation drug approved for use in transplantation field. The Part of the challenge in addressing the potential of rapamycin (or its analogs) as a pro-longevity therapeutic lies in its known clinical risks for adverse side effects. Primary amongst these are metabolic defects that include hyperglycemia, hyperlipidemia, insulin resistance and increased incidence of new-onset Type 2 Diabetes. In healthy rodents, treatment with rapamycin also causes a relatively rapid, dose-dependent impairment of markers of glucose homeostasis (3).

In recent years, there is an explosive epidemic of obesity and diabetes that accelerates aging and its related illnesses. This aspect is detailed by Youssef M K et al in “Diabesity: an overview of a rising epidemic” Nephrol Dial Transplant (2011) 26: 28-35(4). This is negating aging research efforts.

Polakof S in “Diabetic Therapy: Novel Patents targeting the Glucose-Induced Insulin Secretion” Recent Patents on DNA and Gene sequences, 2010, 4:1-9 summarizes evolving new modes of therapies for diabetes (5)

Binder et al in U.S. Patent Application Publication dated Jan. 10 2013 in U.S. 2013/0011857 A1 (6) details the use of recombinant Factor H and its therapeutic formulations for Oxidative stress disease conditions that include variety of age related diseases such as cardiovascular diseases, metabolic syndrome, obesity, autoimmune disease, multiple sclerosis, cancer and conditions caused by cancer treatment, age related macular degeneration, Alzheimer's disease, brain senescence, alcoholic liver disease, ischemic reperfusion injury, diabetic nephropathy, nephritis, acute lung injury, an infectious disease, and an inflammatory condition associated with or caused by one of the foregoing.

Rahiman S and Tantry B. A. in “Nano medicine current trends in diabetes management” J Nanomed Nanotechnol, 2012, 3:5; 1-7 (7) summarizes the application of Nanotechnology and its formulation methods for diabetes.

Two centuries ago, the human average life span was below 40 years. Fortunately, nowadays, the life span rose to approximately 80 years in many developed countries (see details at http://www.mortality.org.)

The United States population is aging at an unprecedented rate with the over 65 populations expected to more than double by 2040. The population 65 and over has increased from 35.5 million in 2002 to 43.1 million in 2012 (a 21% increase) and is projected to increase to 79.7 million in 2040. The over 85 population expected to nearly triple. The 85+ population is projected to increase from 5.9 million in 2012 to 14.1 million in 2040 and the over 100 population expected to more than sextuple (U.S. Administration on Aging;http://www.aoa.gov/Aging_Statistics/future_growth/future_growth.aspx#age).

Genetic and environmental interactions with Factor H cause immune pathogenesis of numerous age related illnesses. This aspect is extensively detailed in the basic science literature http://www.ncbi.nlm.nih.gov/pubmed/? term=Factor+H.

Therapeutic modulation of adverse effects of Factor H therefore have important implications in aging research because it unearths a common pathway of age related illnesses that can be modified in a cost effective way to control age related illnesses, prolong healthy longevity, improve productivity, reduce disabilities and its associated burgeoning health care cost.

Below we detail our invention for the prophylaxis and therapy of Type 2 Diabetes and its complications. This is a model example of the immune pathogenesis of age related illnesses that is due to genetic and environmental interactions with Factor H.

### SUMMARY OF THE INVENTION

It is the first object of the invention to define the physiological role of Factor H in the protection of structure and function of insulin secreting Beta cells in the pancreas.

It is the second object of the invention to define the immune pathogenesis of diabetes and its complications in relation to the genetic and environmental interactions with Factor H.

It is the third object of the invention to target immune pathogenesis of diabetes and its complications by targeting adverse effects of Factor H.

It is the fourth objet of the invention to develop prophylaxis for diabetes and its complications by preventing adverse effects of Factor H.

It is the fifth object of the invention to develop therapy of diabetes and its complications by targeting adverse effects of Factor H.

It is the sixth object of the invention to develop Nano formulated methods for multipronged targeting of immune pathogenesis of diabetes and its complications by combining strategically existing and evolving drugs.

It is also the object of the invention to extend above model of prophylaxis and therapy to prevent major age related illnesses for the extension of healthy longevity.

These and other related objects are achieved by a method for treating a subject suffering from age related illnesses comprising the steps of administering to the subject a compound to down regulate Factor D or Factor H thereby providing prophylaxis and therapy to inhibit age related illnesses.

The administering step comprises one of (i) administering to the subject a compound that primarily inhibits Factor D, (ii) administering to the subject a compound that primarily inhibits Factor H, and (iii) administering to the subject a compound that primarily inhibits Factor D and H. The compound is a functionalized Nano Polymer of less than 100 nm.

The administering step that primarily inhibits Factor D further includes secondary inhibition of a cross talk path of Factor D comprising inhibiting serine proteases and an amplification loop for down regulating C3a-C5a, anaphylotoxins or Acylation Stimulating Protein (ASP) or combinations thereof.

The administering step that primarily inhibits Factor H further includes secondary inhibition of a cross talk path of Factor H comprising down regulating Adrenomedullin (AM), serine proteases or combinations thereof.

The administering step that primarily inhibits Factor H further includes inhibiting the adverse effects of Factor H in degenerative neurological diseases, Age-related Macular Degeneration (AMD), Alzheimer's Disease, Multiple sclerosis or stroke.

The administering step that primarily inhibits Factor H further includes inhibiting the adverse effects of Factor H in diabetic retinopathy, autoimmune uveitis or inflammatory eye diseases.

The administering step that primarily inhibits Factor H further includes inhibiting the adverse effects of Factor H in coronary heart disease, hypertension or atherosclerosis.

The administering step that primarily inhibits Factor H further includes inhibiting the adverse effects of Factor H in oncological diseases, pancreatic cancer, glioblastoma, thyroid cancer, small cell carcinoma of lung, colon cancer or liver cancer.

The administering step that primarily inhibits Factor H further includes inhibiting the adverse effects of Factor H in skin carcinoma, inflammatory diseases of the skin, psoriasis or UV mediated inflammation.

The administering step that primarily inhibits Factor H further includes inhibiting the adverse effects of Factor H in pandemics, influenza A, Ebola or HIV.

The functionalized Nano Polymer comprises Nano polymer Sodium Polystyrene Sulfonate (NSPS). The funtionalized Nano polymer Sodium Polystryrene Sulfonate (NSPS) may be administered by one of oral administration, subcutaneous administration, intravenous administration and local administration as needed. The functionalized Nano polymer Sodium Polystryrene Sulfonate (NSPS) may be administered in formulations including anti-diabetic medications.

The administering step further includes additional inhibition of AM binding protein 1 to stimulate anaphylotoxins, C3a and C5a at tissue injury sites for tissue healing, growth, repair and to maintain telomerase length.

### BRIEF DESCRIPTION OF THE DRAWINGS

The advantages, nature, and various additional features of the invention will appear more fully upon consideration of the illustrative embodiments now to be described in detail in connection with accompanying drawings. In the drawings wherein like reference numerals denote similar components throughout the views.

FIG. 1 is a diagram showing the anatomical location of insulin producing Beta cells in the pancreas.

FIG. 2 is a diagram of islet cell structure and function.

FIG. 3 is a diagram of Factor H and its structure and function

FIG. 4 shows the immune pathogenesis of Diabetes.

FIGS. 5A and 5B are comparative bar graphs of Factor D inhibition with Nano sulfonated Polymer.

FIGS. 6A and 6B are comparative bar graphs of inhibition of Factor H with Nano sulfonated Polymer

### DETAILED DESCRIPTION OF THE PREFERRED EMBODIMENTS

Above objects of invention will be clearer to the practitioners of art by perusing following diagrams in the context of recent advances in Factor H.

These advances can be readily applied to define the physiological role of Factor H and its contribution to the immune pathogenesis of diabetes and its complications as under.

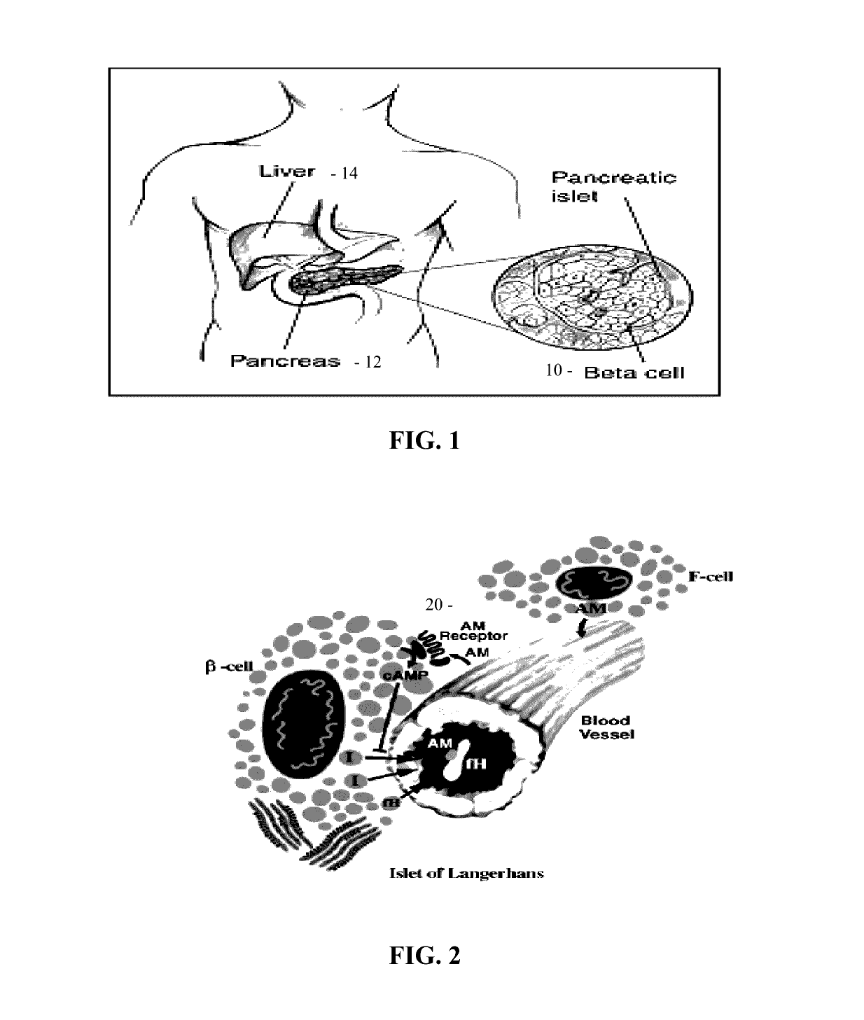

FIG. 1 shows the anatomical Location of Insulin producing Beta cells 10 in the Pancreas. The Pancreas 12 is a gland located in the upper portion of abdomen. It is below the liver 14 and surrounded by duodenum. The endocrine component of the pancreas that is involved in glucose homeostasis is shown as histological structure of pancreas showing the location of insulin producing beta cells.

FIG. 2 shows a new evolving understanding of islet cell structure and function. Adrenomedullin (AM) is a 52 amino acid, multifunctional hormone. It is expressed in many tissues of the human body including the pancreas, where it is mainly localized to the periphery of the islet cells of Langerhans and specifically to the pancreatic polypeptide-expressing cells. The AM receptor 20, a complex formed by calcitonin receptor—like receptor (CRLR) and receptor activity-modifying proteins (RAMPs), and the recently discovered AM-Binding Protein, complement Factor H (FH), are expressed in the insulin-producing Beta cells. The localization of these key elements of the AM system in the endocrine potion of the pancreas implies AM in the control of both normal and altered pancreatic physiologies. AM inhibits insulin secretion both in-vitro (isolated rat islets) and in-vivo (Oral glucose tolerance test in rats) in dose-dependent manner. The addition of FH to isolated rat islets produces a further reduction of insulin secretion in the presence of AM. Furthermore, AM is elevated in plasma with pancreatic dysfunctions such as type 1 or 2 diabetes and insulinoma. In rat, they have shown that AM increases circulating glucose levels where a blocking monoclonal antibody against AM has the opposite effect and improves postprandial recovery. Such experimental evidence implicates AM as a fundamental factor in maintaining insulin homeostasis and normoglycemia and suggests the implication of AM as a possible causal agent in diabetes. Zudaire E et al in “Regulation of pancreatic Physiology by Adrenomedullin and its binding protein” published in Regulatory Peptides 112, 2003, 121-130 (9).

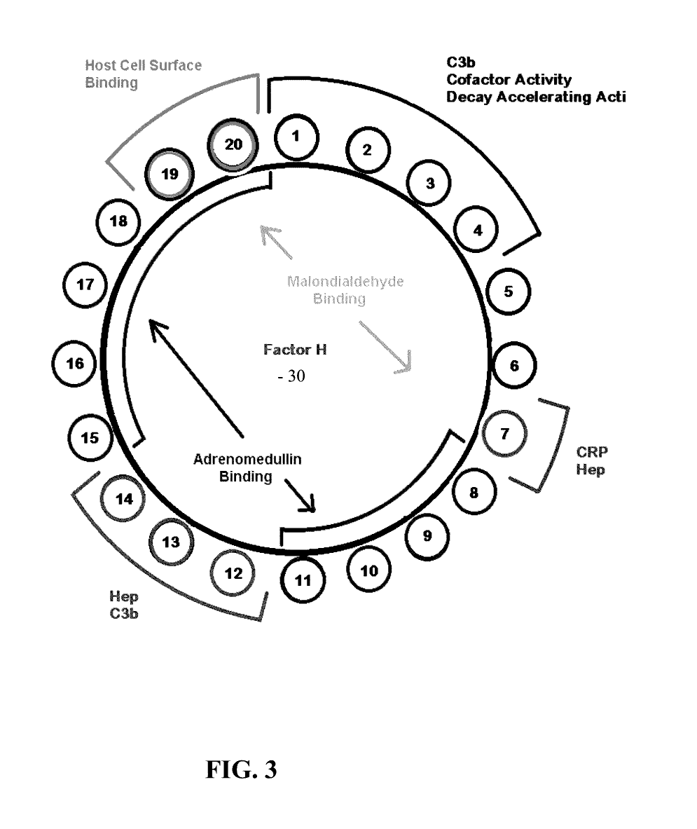

FIG. 3 incorporates recent advances in Structure and functions of Factor H as summarized by Kopp A. et al in “Factor H: A complement Regulator in Health and Disease, and a Mediator of Cellular Interactions” Biomolecules 2012, 2:46-75 (10). It helps to expand above understanding and further explains the structure-function relationship of Islet cells and its functions.

As shown in the FIG. 3, Factor H 30 has a serpentine structure of 150 kD. It circulates in the blood and also binds on the host cell surface. It contains 20 modules of short consens repeats. Each module contains 60 aminoacids. The 20 modules serves as a binding site for various chemicals. Modules can fold upon it self . A circular form of modules help explain structure function of Factor H and its abnormalities or adverse effects clearly as under.

SCR 19-20 bind to host cell surface, fur example, Islet cell surface.

SCR 1-4 contain binding sites for C3b and Cofactor Activities. They work in cooperation with SCR 19-20, to prevent complement amplification loop on islet cells. C3b Bind with Factor H to form C3bH that is further inactivated to iC3b by Cofactor activity of Factor I.

SCR 8-11 and 15-20 are the binding sites for Adrenomedullin on AM binding protein 1 (Factor H modules).

a. AM and AM binding protein 1 (Factor H) work togather to potentiates its inhibitoty effects on amplification loop to prevent immune attack on beta cells.

b. AM and AM Binding protein 1 (Factor H) have inhibitory effects on insulin secretion.

c. AM and AM Binding protein 1 (Factor H) maintain healthy endothelial lining by preventing amplification loop on endothelial surface.

d. AM and AM Binding potein 1 (Factor H) through cyclic AMP and Nitro oxide path, keep vessels in relaxed state and permit blood flow to occur normally.

SCR 7 and 20 are the inkling sites for Malondialdehyde that is a marker for oxidative stress. Both AM and AM binding protein 1 (Factor H) potentiate antioxidative properties.

AM binding Protein 1 abnormalities activate innate inflammatory immune responses that have cross talk functions to increase synthesis of fatty acids, inhibit insulin secretion and compromise capillary circulation. These events compromise islet cell survival, its function, insulin sensitivity, capillary perfusion and metabolic functions.

Based on above we explain the immune pathogenesis of diabetes and its complications due to genetic and environmental interactions with Factor H as under.

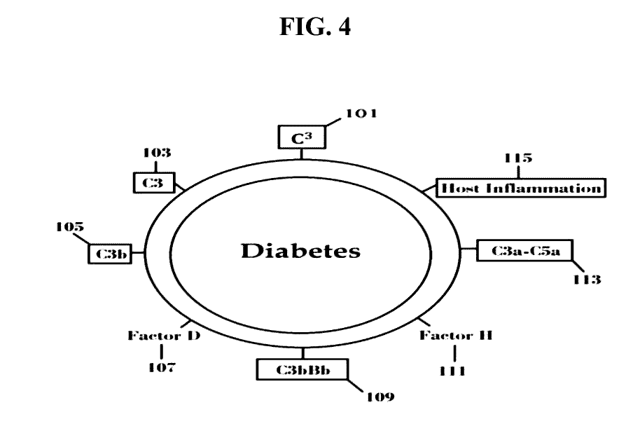

Immune pathogenesis of Type 2 diabetes: FIG. 4 incorporates recent advances in complement system and its role in diabetes and its complications as reviewed by Fujita T. “Extra-immunological role of complement activation in diabetic nephropathy” OA Nephrology, 2013, 25; 1(2), 1-19 (11).

As shown in FIG. 4, the immune pathogenesis of diabetes and its complications are redefined. There are three arms of Complement System (C³), item 101. These include a. classical, b. Lectin based, c. Alternate Complement System. Once activated, the end objective of all three arms of complement system is to form common C3 Molecule, item 103.

In the presence of abnormal glycation and insulin resistance there is over expression of Factor B, C3b, item 105. This activates Factor D, item 107 to form C3bBb Convertase, item 109. This initiates formation of Membrane attack complex through assembly of C5b-C9 Complex. However, there is also over expression of Factor H, item 111 that acts as a negative immune regulator of C3bBb convertase leading to breakdown of C3bBb. (Moreno-Navarette J. M. et al in “Complement Factor H is Expressed in Adipose Tissue in Association With Insulin Resistance” Diabetes, 2010, 59:200-209 (12).

The breakdown products of C3bBb form as C3a-C5a, item 113 and its derivative product acylation stimulating protein (C3adesArg). C3a and C5a are anaphylotoxins and chemotoxins. While, C3adesArg contribute to fat synthesis. When “Serine Protease” of complement system is activated it leads a series of downstream chain reactions. For example, there is constant generation of C3a and C5a fragments. These are chemotactic and anaphylactic fragments. They stimulate Toll Receptors functions of TLR4, TLR2. TLR 6 and TLR 9 in cells. This liberates inflammatory cytokines such as IL-1, IL-6 and TNF Alfa. Inflammatory cytokines, item 115 cause insulin resistance and metabolic syndrome. Maslowska M et al “Targeting the signaling pathway of acylation stimulating protein” J Lipid Res, 2006,47:643-652 (13).

Thus in Type 2 diabetes, there is an increased expression of:

a. Anaphylotoxins such as C3a-C5a: Increased expression of these proteins contributes to inflammation of islet cells and endothelial surface leading to progressive destruction of islet cells.

b. Acylation stimulating protein C3adesArg: This is a breakdown product of C3a that has cross talk effects with Fat cells contributing to increased fatty acid synthesis.

c. Factor H and AM: There is increased expression of Factor H that has inhibitory effects on Insulin secretion. Since Factor H is also binding protein for AM, AM causing insulin resistance potentiates the effect.

d. Factor B: This is increased causing formation of C3bB complex that interact with Factor D or adipsin causing formation of C3bB convertase.

The dysfunction of Alternate complement system due to above events cause islet cell inflammation, ischemic reperfusion injury, decrease secretion of insulin and increased fat synthesis. This precipitates Insulin resistance syndrome, morbid obesity, metabolic syndrome, Nonalcoholic Fatty Liver disease, micro vascular diseases of diabetes affecting eyes, kidneys and nerves as well as macro vascular disease leading to coronary atherosclerosis and other cardiovascular diseases.

Above understandings is in keeping with insulin resistance syndrome and increased cardiovascular diseases seen in following syndromes:

Chen D. et al in “Clinical Review 153: Lipodystrophy in Human Immunodeficiency Virus-infected Patients” The J Clin. Endocrinol. Metab, 2002:87; 4845-4856 (14).

Savage D. B. et al “Complement Abnormalities in Acquired Lipodystrophy Revisited” J ClinEndocrinolMetab94: 10-16, 2009 (15).

Siezenga M. A. et al “Enhanced complement activation is part of the unfavorable cardiovascular risk profile in South Asians” Clinical and Experimental Immunology” 2009, 157:98-103(16).

Segers F M Et Al “ Complement Alternate Pathway Activation in Human Nonalcoholic Steatohepatitis” PLoS ONE, 2014, 9(10); e110053 (17).

Wong H. K. et al in “Adrenomedullin and diabetes” published in World J Diabetes 2014, Jun. 15:5(3); 364-371 (18) further details the role of AM and reviews the role of AM in diabetes.

Cutitta et al in U.S. Pat. No. 6,320,022B1 titled “Adrenomedullin Peptides” granted on Nov. 20, 2001 (19) details the role of AM in diabetes and various cancer lines driving neoplastic proliferation. The method of the invention discloses the monoclonal ab MoAbG6, which neutralizes AM bioactivity and increase insulin secretion fivefold, an effect that was reversed by the addition of synthetic AM.

Many of the versatile properties of AM detailed in above invention is also described in relation to pancreatic cancer and diabetes by Sah R. P. et al in “New insights into pancreatic cancer-induced Para neoplastic diabetes” published in Nat Rev Gastroenterol Hepatol 2013 July: 10(7): 423-433 (20).

Aggarwal G et al in “Adrenomedullin is up regulated in patients with pancreatic cancer and causes insulin resistance in Beta cells and mice” published in Gastroenterology: 2012 December 143(6); 1510-1517 (21) further details the versatile role of AM.

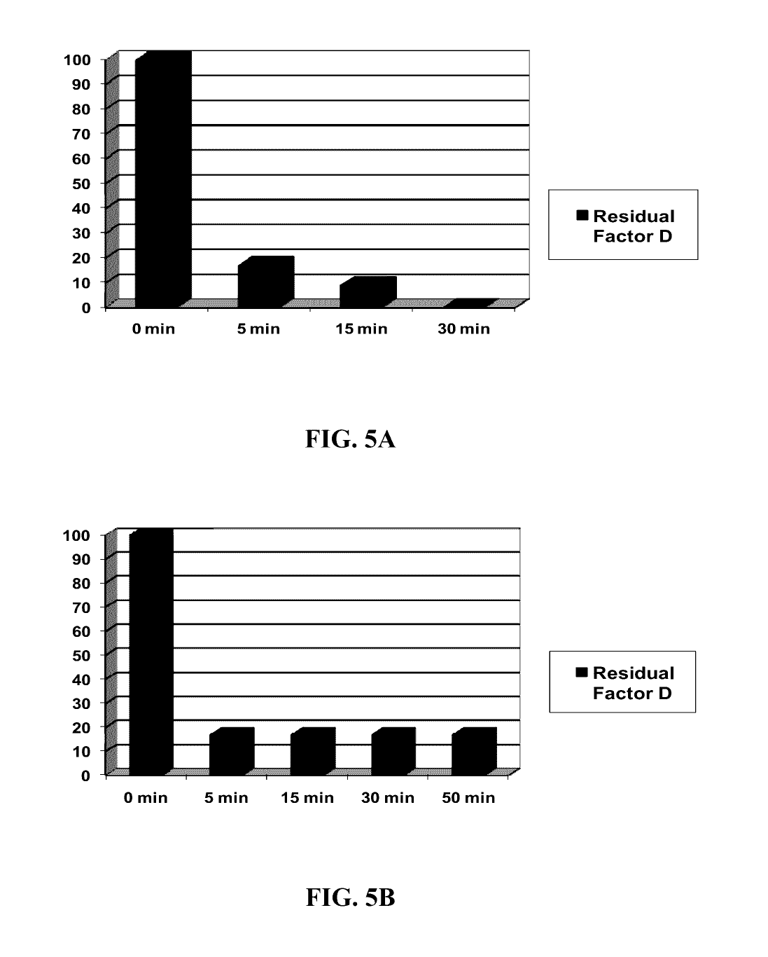

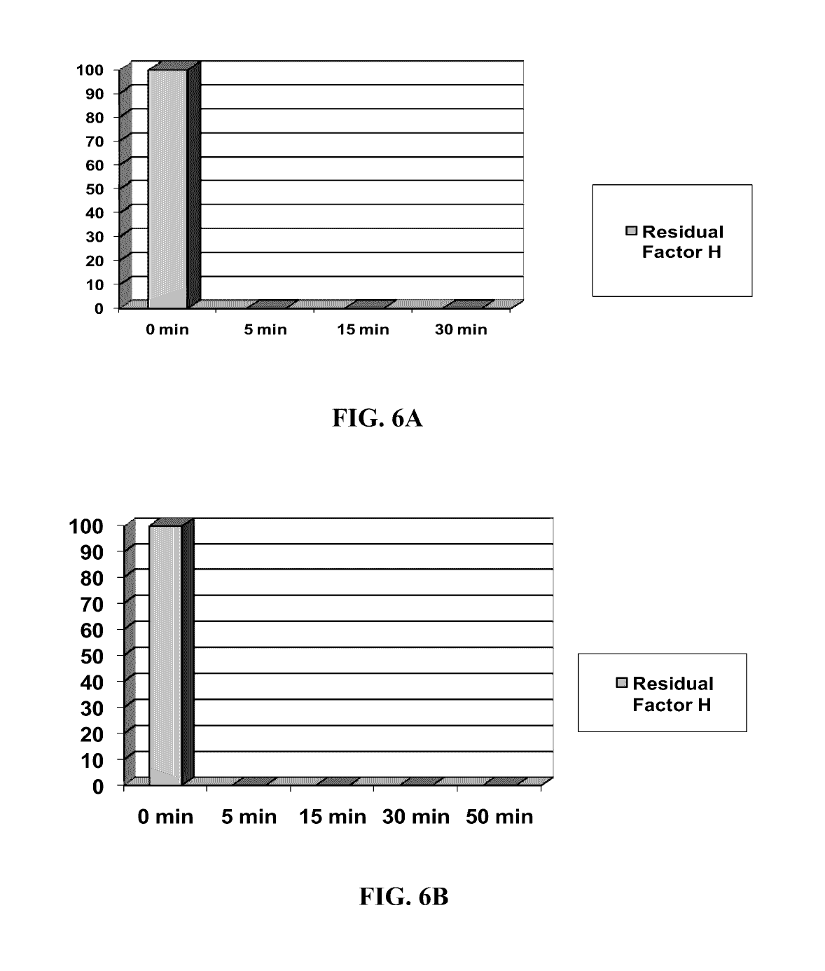

Below in FIGS. 5A and 5B, and in FIGS. 6A and 6B we detail functionalized Nano Polymer of less than 100 nm to inhibit Factor D and H. By inhibiting Factor D and H, many of the adverse effects of insulin resistance syndrome can be inhibited.

Inhibition of Factor D inhibits serine proteases and amplification loop involved in formation of C3a, C5a anaphylotoxins that cross talk with fat to increase triglyceride synthesis. Factor H is tightly bound to AM. Since both, Factor H and AM mutually potentiate the regulatory inhibition of insulin, it is expected that simultaneous inhibition of Factor H with Factor D will reduce AM and like AM antibodies will increase insulin secretion. Protection of islet structure, endothelial function and increase insulin secretion by inhibiting Factor D and Factor H will reduce C3a-C5a and formation of C3adesArg reducing insulin resistance and improve insulin sensitivity.

The Inhibition of Factor D is illustrated in FIG. 5A that shows residual Factor D with NSPS. When in in-vitro experiments fresh Normal Human Serum in 1/10 dilution is contacted with NSPS and incubated for 30 minutes at 37 *C, the drug inhibit Factor D at 60 mg dose.

Compare with FIG. 5B, for in-vitro experiments using fresh Normal Human Serum in 1/10 dilution is contacted with NSPS and incubated for 30 minutes at 37 *C, the drug inhibit Factor D. The Factor D inhibition at 60 mg dose is 85% and sustained over 50 min.

The Inhibition of Factor H is illustrated in FIG. 6A that shows Residual Factor H with NSPS. When in in-vitro experiments fresh Normal Human Serum in 1/10 dilution is contacted with NSPS and incubated for 30 minutes at 37 *C, the drug inhibit Factor H. Thus 100% factor H inhibition in-vitro occurs at 30 mg dose.

Compare with FIG. 6B for in-vitro experiments using fresh Normal Human Serum in 1/10 dilution is contacted with NSPS and incubated for 30 minutes at 37 *C, the drug inhibit Factor H. Thus 100% factor H inhibition in-vitro occurs at 30 mg dose.

Since many of the complications seen in diabetes are related to insulin resistance state, down regulation of Factor D and H in FIGS. 5A and 5B and in 6A and 6B are thought to be of particular value in both prophylaxis and therapy. Thus prophylaxis and therapy of Insulin resistance syndrome is down regulation of Factor D and Factor H. This in turn will down regulate serine proteases, C3a-C5a, ASP and AM. Adverse effects of such therapy can be monitored by simple laboratory tests and tests directed to complement assays and their functions.

Above model of immune pathogenesis of Diabetes and its complications can be readily applied to many age related illnesses. Table 1 details immune pathogenesis of individual age related diseases, These are due to genetic and environmental interactions with Factor H causing its adverse effects.

1. A method for treating a subject suffering from diabetes related illnesses comprising the steps of:
providing a formulation including an anti-diabetic medication and a functionalized Nano Polymer compound of less than 100 nm that inhibits Factor D and Factor H to reduce C3a-C5a and formation of C3adesArg to improve insulin sensitivity ; and
orally administering the formulation to the subject to down regulate Factor D and Factor H thereby providing therapy to treat diabetes related illnesses.

2. (canceled)

3. The method of claim 1, wherein said administering step further includes:
secondary inhibition of a cross talk path of Factor D comprising inhibiting serine proteases and an amplification loop for down regulating anaphylotoxins or Acylation Stimulating Protein (ASP) or combinations thereof.

4. The method of claim 1, wherein said administering step further includes:
secondary inhibition of a cross talk path of Factor H comprising down regulating Adrenomedullin (AM), serine proteases or combinations thereof.

5. (canceled)

6. The method of claim 1, wherein said administering step further includes inhibiting the adverse effects of Factor H in diabetic retinopathy.

7-10. (canceled)

11. The method of claim 1, wherein the functionalized Nano Polymer comprises Nano polymer Sodium Polystyrene Sulfonate (NSPS).

12. (canceled)

13. (canceled)

14. The method of claim 1, wherein said administering step further includes additional inhibition of AM binding protein 1 to stimulate anaphylotoxins, C3a and C5a at tissue injury sites for tissue healing, growth, repair and to maintain telomerase length.
